(19) United States
(12) Patent Application Publication
(10) Pub. No.: US 2007/0055213 A1
(43) Pub. Date: Mar. 8, 2007

(54) FEMININE HYGIENE PRODUCT DISPOSAL SYSTEM AND METHOD

(76) Inventor: Barbara Erekson, Woodland Hills, CA (US)

Correspondence Address: CHRISTIE, PARKER & HALE, LLP, PO BOX 7068, PASADENA, CA, 91109-7068, US

(21) Appl. No.: 11/470,956

(22) Filed: Sep. 7, 2006

Related U.S. Application Data
(60) Provisional application No. 60/715,218, filed on Sep. 7, 2005.
(60) Provisional application No. 60/730,588, filed on Oct. 26, 2005.

(51) Int. Cl. A61F 13/15

(52) U.S. Cl. 604/385.13; 604/385.02

(57) ABSTRACT

A system for disposal of feminine hygiene products includes a dispenser with a dispensing opening and an inner cavity. A bag is disposed within the inner cavity, and sized to hold a feminine hygiene product. In one embodiment, the system also includes the feminine hygiene product. The bag may include a closeable portion for enclosing the feminine hygiene product within the bag. A method of protecting against contamination from a feminine hygiene product includes providing a soiled feminine hygiene product, providing a liquid proof bag sized to enclose the soiled feminine hygiene product, inserting the soiled feminine hygiene product into the bag, and closing the bag to enclose the soiled feminine hygiene product inside the bag.

### CROSS-REFERENCE TO RELATED APPLICATION(S)

This application claims priority to and the benefit of U.S. Provisional Application Nos. 60/715,218, filed on Sep. 7, 2005 and 60/730,588, filed Oct. 26, 2005, which are incorporated by reference as if set forth in full herein.

### BACKGROUND

When faced with the task of disposing of a feminine hygiene product, such as a sanitary napkin or tampon, a woman typically drops the soiled hygiene product directly into a trash receptacle or flushes the product down the toilet. These methods, however, result in contaminated trash receptacles, embarrassing sights and odors, and clogged toilet drains. To avoid these consequences, a woman may even decide to leave the soiled hygiene product in place, which may lead to serious health problems, embarrassing odors, and leakage onto clothing.

A woman may alternatively attempt to wrap the soiled product in toilet tissue before throwing the product into the receptacle. However, the product typically bleeds through the tissue, leading to the same problems of contamination and embarrassing sights and odors. Further, a woman may instead attempt to carry the wrapped, soiled product in her purse until she can find an available or discrete trash receptacle, but this method can lead to even greater contamination, embarrassment, and health problems.

### SUMMARY OF THE INVENTION

A system for disposal of feminine hygiene products includes a dispenser with a dispensing opening and an inner cavity. A bag is disposed within the inner cavity, and sized to hold a feminine hygiene product. In one embodiment, the system also includes the feminine hygiene product. The bag may include a closeable portion for enclosing the feminine hygiene product within the bag.

The closeable portion can create a liquid-proof seal. In various embodiments, the closeable portion may also include an adhesive portion, an interlocking groove portion and a projecting portion, tiable elements, or an outer pocket on the bag that is formed to close an opening of the bag when it is turned inside out.

In some embodiments, the bag is opaque, scented, and/or substantially biodegradable. The bag may be formed of low density polyethylene, and, in some embodiments, has a length of approximately three inches to ten inches and a width of approximately two inches to eight inches. Walls of the bag may be approximately 0.025 mm to 0.15 mm thick. The bag may be releasably attached to a plurality of bags through a perforated material or through other means. The plurality of bags may be rolled together with the bag into a roll, disposed within the inner cavity of the dispenser, and coupled to the dispenser such that the bag may be dispensed through the dispensing opening.

The dispenser may further include a latch coupled to the bag to dispense the bag through the dispensing opening. The dispenser may also be formed as a bag.

A method of protecting against contamination from a feminine hygiene product includes providing a soiled feminine hygiene product, providing a liquid proof bag sized to enclose the soiled feminine hygiene product, inserting the soiled feminine hygiene product into the bag, and closing the bag to enclose the soiled feminine hygiene product inside the bag.

The method may further include providing a trash receptacle and depositing the closed bag into the trash receptacle. The closing may include sealing the bag. In one embodiment, the method also includes providing a dispenser surrounding the bag and dispensing the bag from the dispenser.

### BRIEF DESCRIPTION OF THE DRAWINGS

FIG. 1a is a partial cut-out and exploded perspective view of one embodiment of a system for disposal according to the invention.

FIG. 1b is a front perspective view of the embodiment shown in FIG. 1a in a closed position.

FIG. 1c is a front perspective view of the dispenser shown in FIGS. 1a and 1b in an open position with an alternate embodiment of a bag according to the invention.

FIG. 2a is a bottom perspective view of another embodiment of a system for disposal according to the invention.

FIG. 2b is a front perspective view of the embodiment shown in FIG. 2a.

FIG. 2c is a side perspective view of a roll of bags according to one embodiment of the invention.

FIG. 3 is a front perspective view of another embodiment of a system for disposal according to the invention.

FIG. 4 is a front perspective view of one embodiment of a bag according to the invention.

FIG. 5a is a front perspective view of another embodiment of a bag according to the invention.

FIG. 5b is a front perspective view of the bag shown in FIG. 5a in a first stage of being closed.

FIG. 5c is a front perspective view of the bag shown in FIGS. 5a and 5b in a second stage of being closed.

FIG. 5d is a rear perspective view of the bag shown in FIGS. 5a-5c in a closed position.

FIG. 5e is a front perspective view of the bag shown in FIGS. 5a-5d.

FIG. 6 is a schematic diagram of one embodiment of a method according to the invention.

FIG. 7 is a front perspective view of another embodiment of a system for disposal according to the invention.

FIG. 8 is a perspective view of yet another embodiment of a system for disposal according to the invention.

FIG. 9 shows a plan view and a side perspective view of the embodiment shown in FIG. 8.

### DETAILED DESCRIPTION OF THE INVENTION

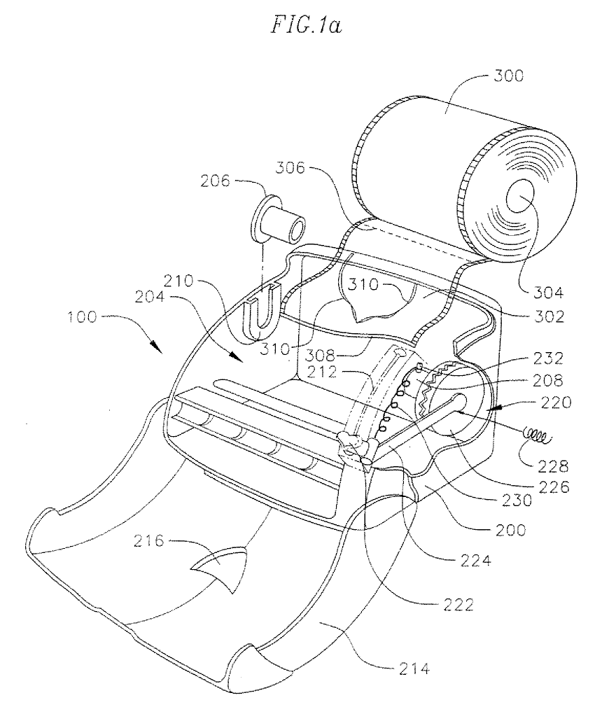

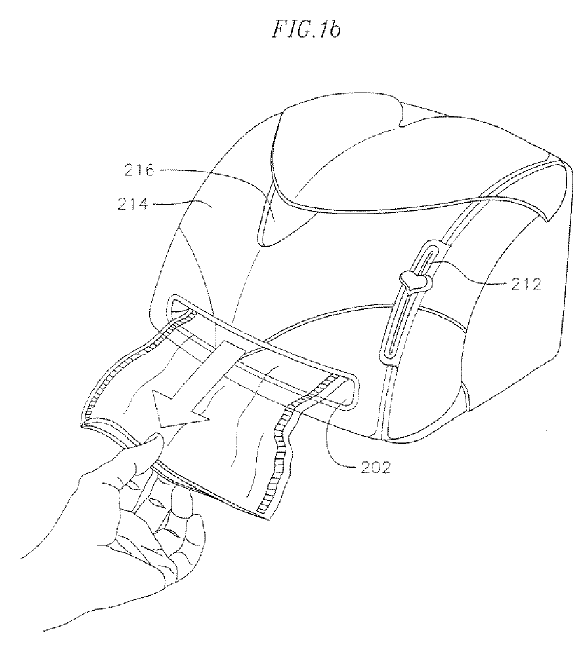

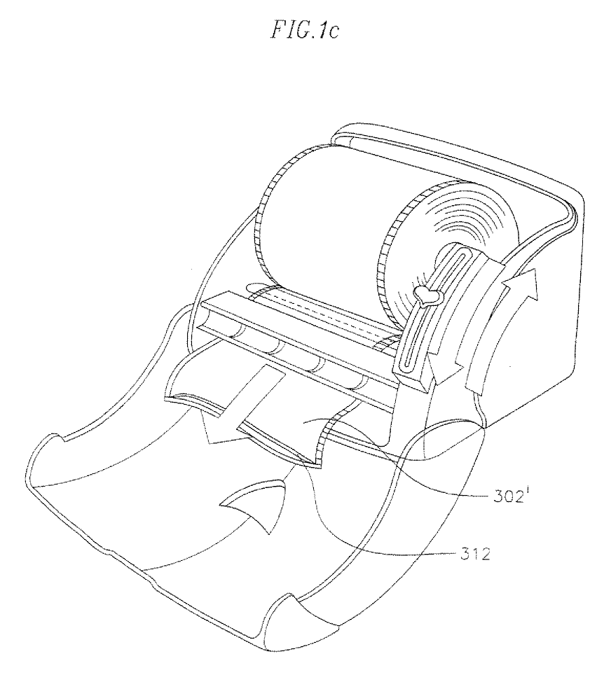

As can be seen in FIGS. 1a and 1b, a system for disposal of feminine hygiene products 100 includes a dispenser 200 with a dispensing opening 202 and an inner cavity 204. The dispenser 200 also includes a slit 212 and a viewing window 216 in its hinged, front face 214.

A roll 300 of bags 302 with a central opening 304 is disposed within the inner cavity 204. The central opening 304 of the roll of bags 300 engages with protrusions 206, 208 on opposite lateral sides of the inner cavity 204. One protrusion 206 rotates freely within a groove 210 fixed on the dispenser 200. The other protrusion 208 extends from a latch mechanism 220 and engages with force locking engagement with the central opening 304 of the roll 300.

The latch mechanism 220 in this embodiment includes a handle 222 that extends through the slit 212 in the front face 214 of the dispenser 200. The handle 222 is rigidly connected to an arm 224 fixed to a rotatable, toothed catch gear 226. A compression spring 228 is disposed between the arm 224 and the catch gear 226 to pushes the teeth of the catch gear 226 into engagement with teeth of the protrusion 208. A tension spring 232 couples the distal end of the arm 224 to the protrusion 208 to pull the handle 222 upward.

As shown in FIGS. 1a-1c, as the handle 222 is pulled against the force of the tension spring 230 toward the bottom of the slit 212, the arm 224 rotates the catch gear 226 so that it is not in toothed engagement with the protrusion 208. As the handle 222 is brought back up due to the force of the tension spring 230, however, the teeth of the catch gear 226 engage with the teeth of the protrusion 208 to rotate the protrusion 208. The roll 300 is thus rotated through force-locking engagement with the protrusion 208, whereby a single is bag 302 is dispensed through the dispensing opening 202, and can be grasped and detached from the roll 300 by tearing its perforations 306.

The bag 302 shown in FIG. 1a has a closeable portion 310 adjacent to the opening 308 of the bag 302. One closeable portion includes at least two tiable elements 310, which, after a soiled feminine hygiene product is placed within the bag 302, the tiable elements 310 are tied tightly together to close the opening 308 of the bag 302.

Alternatively, as shown in FIG. 1c, a bag 302′ may have a closeable portion 312 that includes a groove portion and a projecting portion facing the groove portion and interlockable with the groove portion, such as a Zip-Lock™-type closure.

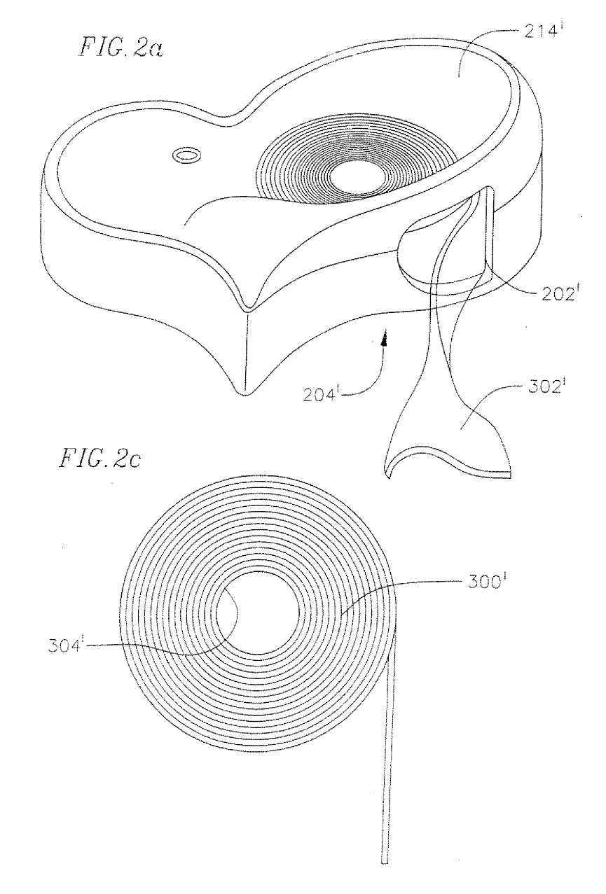

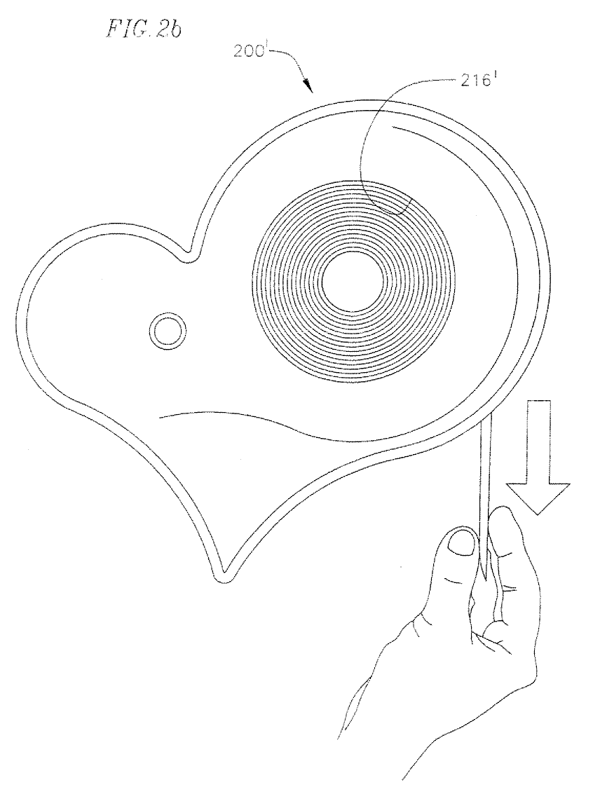

As shown in FIGS. 2a-2c, another embodiment of a system for disposal of feminine hygiene products 100′ includes a dispenser 200′ with a dispensing opening 202′ and an inner cavity 204′. The dispenser 200′ also includes a viewing window 216′ in its front face 214′.

A roll 300′ of folded bags 302′ is disposed within the inner cavity 204′. A folded bag 302′ extending out of the dispensing opening 202′ can be grasped, pulled, and its perforations connecting it with the other bags can be torn. In this embodiment, the roll 300′ of bags 302′ sits and can rotate freely within a substantially cylindrically shaped portion of the inner cavity 204′. However, it is also within the scope of the invention to provide one or more protrusions in the inner cavity 204′ to engage the central opening 304′ of the roll 300′.

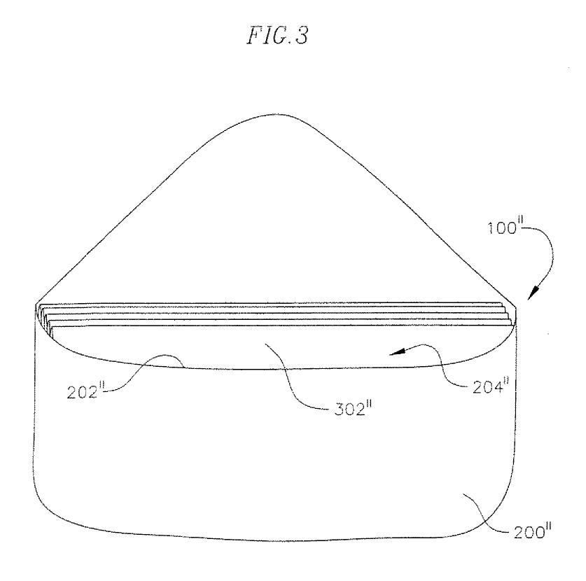

FIG. 3 depicts yet another embodiment of a system for disposal of feminine hygiene products 100″ including a dispenser 200″ with a dispensing opening 202″ and an inner cavity 204″. The dispenser 200″ is in the shape of a bag and is in a size convenient for carrying in a pocket or a purse.

Folded bags 302″ are disposed within the inner cavity 204″ of the dispenser 200″. Each folded bag 302″ may be grasped individually from the dispenser 200″ and used.

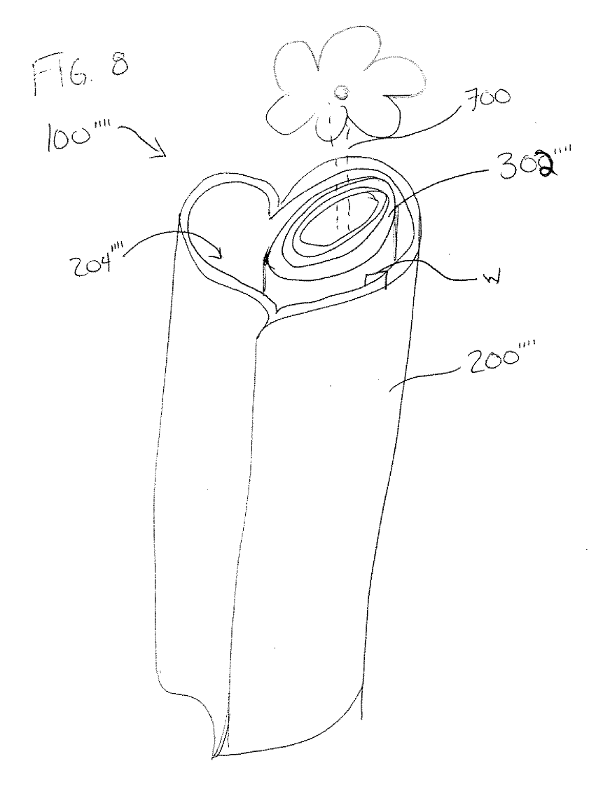

As shown in FIG. 7, the disposal system 100′″ may include a dispenser 200′″ in the form of a mountable housing with screw holes 201. The dispenser 200′″ includes folded bags 302′″, similar to those described above, within its inner cavity 204′″.

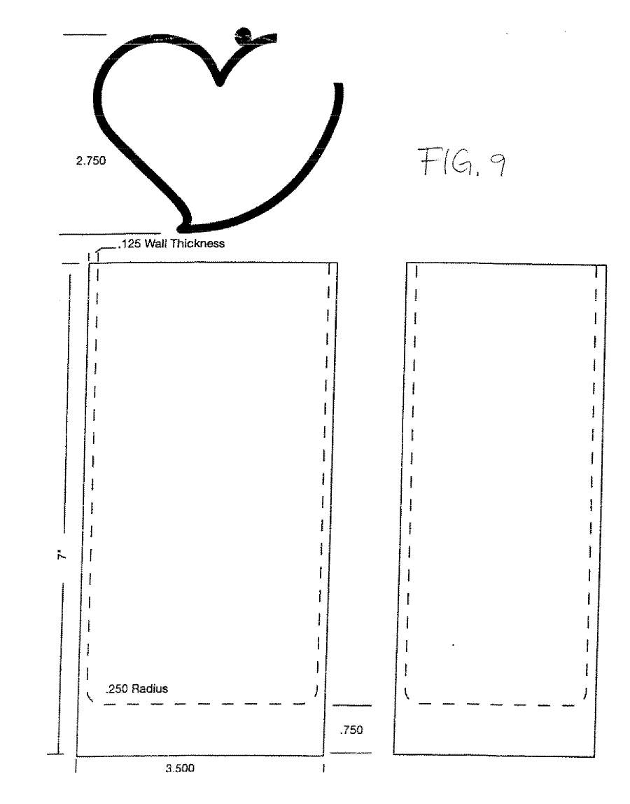

As shown in FIG. 8, the disposal system 100″″ may include a dispenser 200″″ in the form of a vase. In this embodiment, the shape of the vase from a top view is in the shape of the heart, but the invention is not limited thereto. A roll of bags 302″″ is located within the inner cavity 204″″. A silk flower 700 is also included in this embodiment, and fits within an opening in the roll of bags 302″″. In this embodiment, the vase has a width w of the side wall of approximately 0.125 inch, an approximately 0.250 inch inner radius, and an approximate height of 7 inches. However, the invention is not limited thereto.

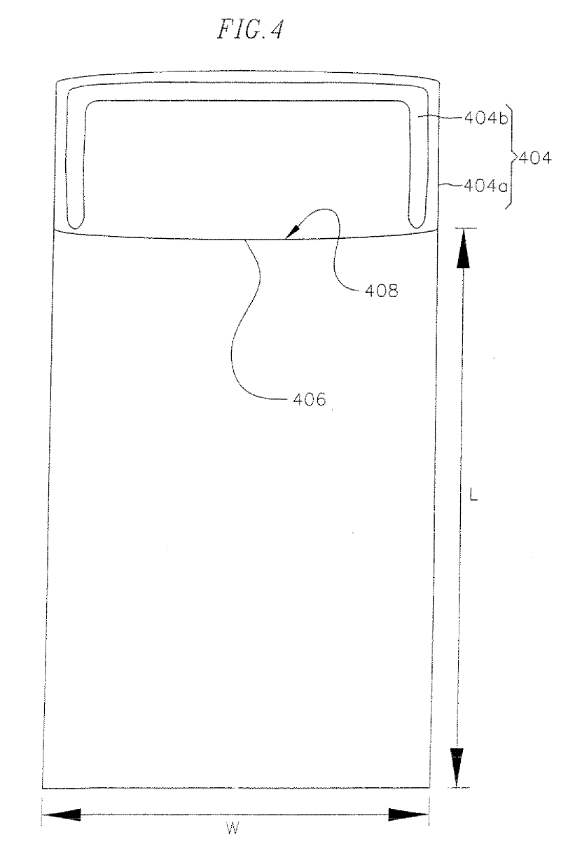

Another embodiment of a bag 402 is shown in more detail in FIG. 4. The bag 402 includes a closeable portion 404 for enclosing a feminine hygiene product within the bag. In this embodiment, the closeable portion 404 includes a flap 404a with an adhesive strip 404b extending beyond an edge 406 of the bag opening 408. The adhesive strip 404b in this embodiment extends around the periphery of the flap 404a, but it is also within the scope of the invention for the strip to only extend in one direction on the flap. The adhesive strip 404b may be covered by release paper until ready to use. When the flap 404a is folded over the bag opening 406, the adhesive 404b adheres to the edge 406 to create a liquid-proof seal.

In this embodiment, the bag 402 is opaque and scented. These features can reduce possible embarrassment if the bag must be set in an open receptacle or carried in a purse or through a public place to be disposed of in an off-site receptacle. The bag 402 in this embodiment is made from a biodegradable material, such as low density polyethylene (LDPE) with a biodegradable additive, such as an oxo-biodegradable additive available from EPI™ called TDPA.

The length L of the bag is approximately three to ten inches, and its width W is approximately two to eight inches. In this embodiment, the length L is eight inches and the width W is six inches. The flap 404 extends approximately one inch from the edge 406. However, one skilled in the art will understand that the length, width, and extension of the flap may be modified to securely contain any size sanitary napkin or tampon. The thickness of each of the walls of the bag 402 is approximately 0.025 mm to 0.15 mm. The thickness may be varied according to the material of the bag so that the bag is sufficiently strong to resist tears or leaks during its use.

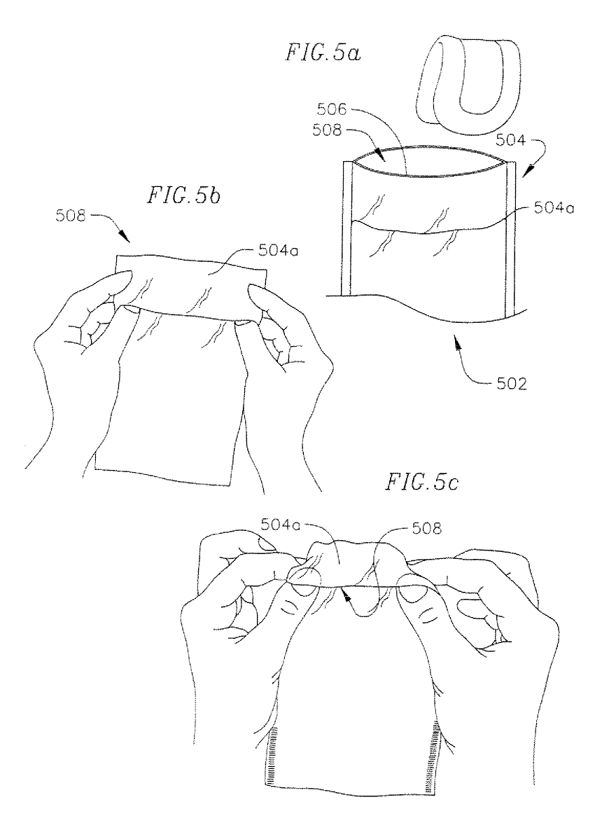

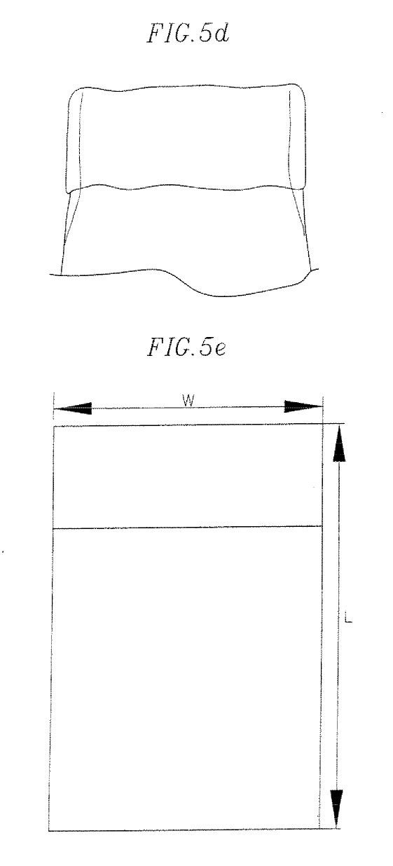

FIGS. 5a-5c show a third embodiment of a bag 502 according to the invention. The bag 502 includes a closeable portion 504 with an outer pocket 504a opening downward and fixed to a rear side of the bag 502. The non-opening side of the outer pocket 504a forms an edge 506 of the opening 508 of the bag 502.

To close the opening 508 of the bag 502, a user slides his or her fingers into the opening of the outer pocket 504a after a soiled feminine hygiene product has been placed inside the bag 502 as shown in the front view of FIG. 5b. The outer pocket 504a is then folded inside out over the opening 508 of the bag 502, as shown in the front view of FIG. 5c and the rear view of FIG. 5d. Adhesive may also be added to the folded edge of the outer pocket 504a so that, when folded inside out, the outer pocket 504a adheres to the front of the bag 502 to seal the opening 508.

As shown in FIG. 5e, the bag 502 has a length L of approximately six inches and a width W of approximately four inches. The outer pocket 504a is approximately one inch deep.

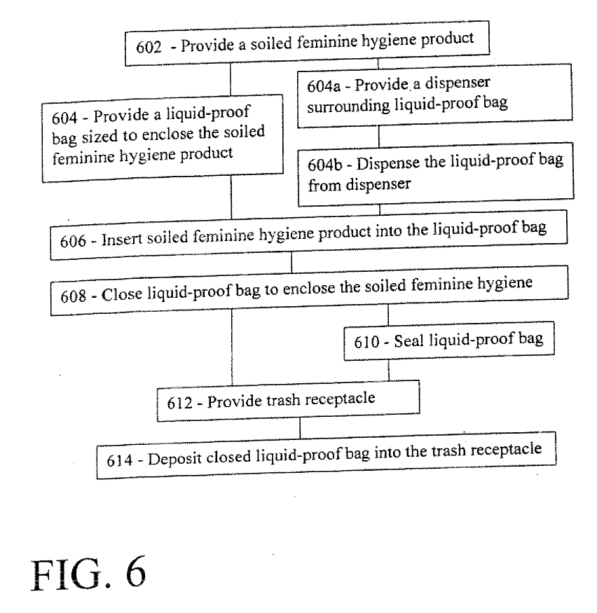

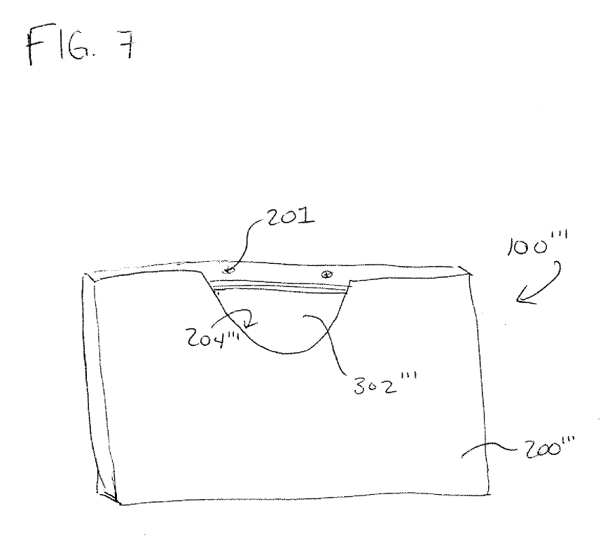

A method of protecting against contamination from a feminine hygiene product is shown in FIG. 6. The method includes providing a soiled feminine hygiene product 602 and a liquid-proof bag 604 sized to enclose the soiled feminine hygiene product. Providing the liquid-proof bag 604 may include providing a dispenser surrounding the bag 604a and dispensing the bag from the dispenser 604b. The soiled feminine hygiene product is inserted into the bag 606. The bag is then closed 608 and, in one embodiment, sealed 610 to enclose the soiled feminine hygiene product inside the bag. A trash receptacle can also be provided 612 and the closed bag may be deposited into the trash receptacle 614.

What is claimed is:

1. A system for disposal of feminine hygiene products comprising:
a dispenser having a dispensing opening and an inner cavity; and
a bag disposed within the inner cavity, and sized to hold a feminine hygiene product.

2. The system of claim 1, further comprising the feminine hygiene product.

3. The system of claim 1, wherein the bag includes a closeable portion for enclosing the feminine hygiene product within the bag.

4. The system of claim 3, wherein the closeable portion can create a liquid-proof seal.

5. The system of claim 3, wherein the closeable portion comprises an adhesive portion.

6. The system of claim 3, wherein the closeable portion comprises a groove portion and a projecting portion facing the groove portion and interlockable therewith.

7. The system of claim 3, wherein closeable portion comprises at least two tiable elements.

8. The system of claim 3, wherein the closeable portion comprises an outer pocket on the bag, the outer pocket formed to close an opening of the bag when it is turned inside out.

9. The system of claim 1, wherein the bag is opaque.

10. The system of claim 1, wherein the bag is scented.

11. The system of claim 1, wherein the bag is substantially biodegradable.

12. The system of claim 1, wherein the bag comprises low density polyethylene.

13. The system of claim 1, wherein the bag has a length of approximately three inches to ten inches and a width of approximately two inches to eight inches.

14. The system of claim 1, wherein walls of the bag are approximately 0.025 mm to 0.15 mm thick.

15. The system of claim 1, wherein the bag is releasably attached to a plurality of bags.

16. The system of claim 15, wherein the bag is attached to the plurality of bags by a perforated material.

17. The system of claim 15, wherein the plurality of bags are rolled together with the bag into a roll.

18. The system of claim 17, wherein the roll is within the inner cavity of the dispenser and coupled to the dispenser such that the bag may be dispensed through the dispensing opening.

19. The system of claim 1, wherein the dispenser further comprises a latch coupled to the bag to dispense the bag through the dispensing opening.

20. The system of claim 1, wherein the dispenser is a bag.

21. A method of protecting against contamination from a feminine hygiene product comprising:
providing a soiled feminine hygiene product;
providing a liquid proof bag sized to enclose the soiled feminine hygiene product;
inserting the soiled feminine hygiene product into the bag;
closing the bag to enclose the soiled feminine hygiene product inside the bag.

22. The method of claim 21, further comprising:
providing a trash receptacle; and
depositing the closed bag into the trash receptacle.

23. The method of claim 21, wherein the closing comprises sealing the bag.

24. The method of claim 21, further comprising:
providing a dispenser surrounding the bag; and
dispensing the bag from the dispenser.
